(19) United States
(12) Patent Application Publication
(10) Pub. No.: US 2020/0046677 A1
(43) Pub. Date: Feb. 13, 2020

(54) FORMULATIONS AND METHOD FOR TREATING SEX HORMONE LOSS IN SPAYED AND NEUTERED ANIMALS

(71) Applicants: Phillip E. Schoenwetter, Rancho Palos Verdes, CA (US); Robin Coon, Santa Rosa, CA (US)

(72) Inventors: Phillip E. Schoenwetter, Rancho Palos Verdes, CA (US); Robin Coon, Santa Rosa, CA (US)

(21) Appl. No.: 16/659,869

(22) Filed: Oct. 22, 2019

Related U.S. Application Data
(63) Continuation-in-part of application No. 14/550,859, filed on Nov. 21, 2014, now abandoned.
(60) Provisional application No. 61/907,559, filed on Nov. 22, 2013.

(51) Int. Cl. A61K 31/353; A61K 31/198; A61K 31/7048; A61K 36/42

(52) U.S. Cl. CPC A61K 31/353; A61K 36/42; A61K 31/7048; A61K 31/198

(57) ABSTRACT

A pet hormone loss supplement composition to mitigate sex hormone loss of mammal pets that have been spayed or neutered. This proprietary formulation is comprised of a specific combination of well-known supplements or food that are generally regarded as safe (GRAS) and serendipitously share either phyto estrogen or phyto androgen properties of sufficient magnitude so as to be considered either a Selective Estrogen Receptor Modulator (SERM) or Selective Androgen Receptor Modulator (SARM) activity. These individual supplements of SERM and SARM properties (a novel and unique combination to maximize the number of hormone receptor site activation) provide a similar and sufficient array of sex hormone mimic activity so as to constitute a functional sex hormone supplement; helping to blunt or eliminate many hormone deficiency effects resulting from spaying or neutering and thus improve health span and possibly life span by more nearly replicating a natural hormone milieu.

### CROSS REFERENCE TO RELATED APPLICATIONS

This application is a continuation-in-part of U.S. patent application Ser. No. 14/550,859, filed Nov. 21, 2014, which claims the benefit of U.S. Provisional Application No. 61/907,559 filed Nov. 22, 2013, both of which are incorporated herein by reference in their entireties.

### FIELD OF THE INVENTION

The present invention relates to novel formulations for mitigating the unintended adverse consequences of sex hormone loss from spay and neutering. Sex hormone loss impacts both genders of all mammals, i.e., dogs, cats, people, goats. In humans we call this menopause or andropause and is known to accelerate the aging process. The goal is to enhance and prolong pets' health span and possibly life span by more nearly matching a normal functional hormone milieu.

Well known natural compounds supplements and food contents each affect a multiplicity of metabolic pathways. In addition to antioxidant and nutritional properties, a few currently consumed natural products also have proven sex hormone receptor modulation capabilities.

This formulation is heavily weighted with natural compounds known to be sex hormone receptor modulator capable. These compounds are:

1. Genistein (various iterations), selective estrogen receptor modulator (SERM).

2. Icariin (various iterations), selective androgen receptor modulator (SARM)/selective estrogen receptor modulator (SERM).

3. Bitter gourd (Momordica charantia, extract, elixirs, charantin), selective androgen receptor modulator (SARM).

4. N-acetyl cysteine (major contributor to glutathione antioxidant pathway).

Spay and neutering alters or removes sex organs, but takes sex hormones with it as well. This is known to adversely affect neurocognitive, cardiovascular, musculoskeletal, and genitorurinary systems. This proprietary combination of natural ingredients is intended to help mitigate these unintended consequences of hormone disruption.

### BACKGROUND OF THE INVENTION

Utilization of compounds or re purposing sex hormone analogs is largely ignored in the pet food/supplement industry. The implications of hormone loss from spaying and neutering and the need to mitigate this loss is a fact that has been hiding in plain site for decades.

### SUMMARY OF THE PREFERRED EMBODIMENTS

In accordance with a first aspect of the present invention there is provided a natural sex hormone loss treatment composition comprising one or more amino acids, a genistein alternative, an icariin alternative, and a Selective Androgen Receptor Modulator (SARM) for treating animals that have been spayed or neutered, in amounts effective to mitigate sex hormone loss in animals.

In accordance with another aspect of the present invention there is provided a method for mitigating the adverse consequences of sex hormone loss resulting from spaying and neutering comprising having the spayed or neutered animal treated with a composition comprising one or more amino acids, a genistein alternative, an icariin alternative, and a SARM, in amounts effective to mitigate sex hormone loss in the treated animal. One goal of the present invention is to enhance and prolong the health span and possibly lifespan of pets by more nearly matching a normal functional hormone milieu.

The invention, together with additional features and advantages thereof, may be best understood by reference to the following description.

### DETAILED DESCRIPTION OF THE PREFERRED EMBODIMENTS

The following description is illustrative and is not to be construed as limiting. Numerous specific details are described to provide a thorough understanding of the disclosure. However, in certain instances, well-known or conventional details are not described in order to avoid obscuring the description. References to one or another embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of compositions, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

In a preferred embodiment of the present invention, the pet sex hormone loss treatment composition may comprise (a) one or more amino acids, preferably those that limit the production of glutathione, (b) a genistein alternative selected from the group consisting of SERMs, genistein (a SERM phytoestrogen), aglycon genistine, hormone receptive modulators, phosphordiesterase (PDE) inhibitors of types 1, 2, 3 and 4, anti-neoplastic tyrosine kinase inhibitors, selective estrogen receptor modulator type beta, and combinations thereof; (c) an icariin alternative selected from the group consisting of icarosides 1 or 2, PDE-4 inhibitors, PDE-5 inhibitors, derivatives of the epimedium species including icarrocides I and II, and combinations thereof, and (d) at least one Selective Androgen Receptor Modulator (SARM) including Momordica charantia L. aka wild bitter gourd (“MC”), a SARM phytoandrogen.

The dosage is based upon the animals weight. In general, from 1 mg/pound to 20 mg/pound, preferably from 1.5 mg/pound to 15 mg/pound, and more preferably from 1.5 mg/pound to 10 mg/pound, of (a) one or more amino acids, preferably those that limit the production of glutathione; and from 0.25 mg/pound to 25 mg/pound, preferably from 0.5 mg/pound to 15 mg/pound, and more preferably from 0.05 mg/pound to 10 mg/pound, of (b) a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, selective estrogen receptor modulator (SERM) type beta, and combinations thereof, and from 0.25 mg/pound to 20 mg/pound, preferably from 2 mg/pound to 10 mg/pound, and more preferably from 1.5 mg/pound to 5 mg/pound, of (c) an icariin alternative selected from the group consisting of icarosides 1 or 2, PDE-4 inhibitors, PDE-5 inhibitors, derivatives of the epimedium species including icarrocides I and II, and combinations thereof; and from 1 mg/pound to 20 mg/pound, preferably from 1.5 mg/pound to 15 mg/pound, and more preferably from 1.5 mg/pound to 10 mg/pound, of (d) at least one Selective Androgen Receptor Modulator (SARM) including Momordica charantia L. aka wild bitter gourd (“MC”).

MC is a plant, and although the entire plant can be used, the different parts of the plant can be used either separately or combined, i.e., the root, leaf, seeds, fruit, flower or combinations thereof The plant and plant parts can be used either fresh or in a dried form. The active ingredients in the plant and plant parts can be extracted to form an extract by using any number of traditional solvents (e.g., water, benzene, ethanol, carbon dioxide, petroleum ether (e.g., butane, hexane) and others. Preferably the fruit and/or seed parts are used either fresh, dry or as an extract, and more preferably a blend of MC fruit and MC seed extracts are used.

Preferably, the amino acid is N-acetyl cysteine, the genistein alternative is genistein or aglycon geninstein, the icariin alternative is icariin, and the SARM is MC.

More preferably, the pet sex hormone loss treatment composition may further include resveratrol. Further preferably, the pet sex hormone loss treatment composition may include lutein or vitamin A in addition to resveratrol. Optimally, the pet sex hormone loss treatment composition may include one or more ingredients selected from the group consisting of resveratrol, lutein, vitamin A, vitamin E, vitamin C, selenium, alpha lipoic acid and combinations thereof. Alternatively, the pet sex hormone loss treatment composition may include horny goat weed extract to provide icariins, d-alpha topopheryl succinate as vitamin E, calcium ascorbate as the source of vitamin C, L-selenomethionine as the source of selenium, beta carotene as the source of vitamin A, and polygnonium cuspidatum as the source of resveratrol.

The pet sex hormone loss treatment composition may be provided to a spayed or neutered animal in any dosage form for the animal to ingest or intake. Preferably it is incorporated into pet food or pet treats. But it can also be sprinkled on pet food or be provided to the pet in the form of a tablet, pill, powder or granules. Alternatively, the dosage may be a liquid dosage form including, but not limited to, emulsions, suspension and spray.

It should be understood that when various dosage forms are utilized to provide the pet sex hormone loss treatment composition to an animal, with a preferred daily dosage, the amounts of the active ingredients of the composition taken on a daily basis are the same, while the weight percentages of the ingredients may vary from dosage form to dosage form. Depending on different dosage forms, the pet sex hormone loss treatment composition may further include a filler, binder, agent or diluter inactive substances selected from the group consisting of microcrystalline cellulose, silicon dioxide, magnesium stearate, caramel and combinations thereof.

Preferably, the pet sex hormone loss treatment composition may further include on a daily basis depending upon the weight of the animal, from about 5 mg to about 200 mg of resveratrol, more preferably, from about 10 mg to about 100 mg of resveratrol, and even more preferably about 12.5 mg of resveratrol, or about 12.5 mg of polygonium cuspidatum; from about 0.5 mg to about 10 mg of lutein, preferably, from about 1 mg to about 5 mg of lutein, and more preferably about 1.25 mg of lutein; from about 250 IU to about 5000 IU of vitamin A, more preferably, from about 500 IU to about 2500 IU of vitamin A and even more preferably about 500 IU of vitamin A, or about 500 IU of beta carotene; from about 30 IU to about 1500 IU of vitamin E, more preferably, from about 60 IU to about 600 IU of vitamin E, and even more preferably, about 30 IU of vitamin E, or about 30 IU of d-alpha topopheryl succinate; from about 25 mg to about 1000 mg of vitamin C, more preferably, from about 50 mg to about 500 mg of vitamin C, and even more preferably about 62.5 mg of vitamin C, or about 62.5 mg of calcium ascorbate; from about 10 micrograms to about 100 micrograms of selenium, more preferably, from about 25 micrograms to about 50 micrograms of selenium, and even more preferably, about 25 micrograms of selenium, or about 25 micrograms of L-selenomethionine; and from about 2 mg to about 200 mg of alpha-lipoic acid; more preferably from about 5 mg to about 100 mg of alpha-lipoic acid, and even more preferably, about 5 mg of alpha-lipoic acid. In a preferred embodiment of the method of the present invention, the pet sex hormone loss treatment composition may be administered to an animal to mitigate sex hormone loss. The method comprises providing an pet sex hormone loss treatment composition in an ingestible form, and causing the animal to ingest or intake an pet sex hormone loss treatment composition comprising one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof and an icariin alternative selected from the group consisting of icarosides 1 or 2, PDE-4 inhibitors, PDE-5 inhibitors, derivatives of the epimedium species including icarrocides I and II, and combinations thereof. Preferably, the pet sex hormone loss treatment composition may be administered at an effective dosage and frequency. More preferably, the preferred dosage and the frequency of administer a preferred dosage may also be altered for the purposes of remedying or treating various degrees of sexual hormone loss. Optimally, the relative percentage weight of each ingredients of the pet sex hormone loss treatment composition may also be altered to enhance the known effectives of the ingredients. The effect of the pet sex hormone loss treatment composition may be quantified by tests well known to those of skill in the art. For example, changes in any of the following can be monitored periodically before and after treatments by known tests: muscle mass, osteo (bone) density, e.g., bone mineral density (BMD), bone calcium density (BCD) and castration elevated serum osteocalcin levels followed by histomorphometric analysis of osteoporosis bone remodeling, urinary bladder control, e.g., measurement of bladder urethral sphincter tightness and pituitary hormone receptors, neuro-cognitive bio markers of cognitive function and cognitive dysfunction, cardiovascular, joint flexibility, weight control and periodontal (teeth) disease. Changes in various bio markers can be measured, e.g., calcitonin and other bio chemical markers of bone absorption, or muscle strength, muscle mass and bone density of teeth and bones.

Optimally, the relative percentage weight of each ingredients of the pet sex hormone loss treatment composition may also be altered to enhance the known effectives of the ingredients. For example, icariin may be included for treatment for lung inflammation, asthmatics and Chronic Obstructive Pulmonary (COPD). For another example, icariin, both an pet sex hormone loss treatment and a neurotrope, increasing osteoblasts in castrated animals with diminished osteoblasts, may be included to raise Bone Mineral Density (BMD) along with genistein. For yet another example, icariin, genistein and resveratrol may be included for the preservation of cognitive function of an animal. For still yet another example, predosing of the pet sex hormone loss treatment compositions may affect the Reactive Oxygen Species (ROS) release of ethanol, a break down and the diminishment of cognitive loss from some anesthetics as well as acute ethanol consumption.

In a preferred embodiment of the present invention, the pet sex hormone loss treatment composition may be administered to spay/neutered animals such as cats and dogs for the purposes of increasing the health and life span of the spayed/neutered animals to address the unintended physiologic consequences of sex hormonal loss arising from spaying and neutering. The use of the pet sex hormone loss treatment composition may delay and mitigate arthropothy and arthritis in spayed/neutered animals by maintaining bone density from the osteoblastic effects of the genistein and icariin. The use of the pet sex hormone loss treatment composition may further increase dental and periodontal health by maintaining bone mass of alveolar and mandibular bones for reasons stated above. The use of the pet sex hormone loss treatment composition may also promote health bladder function and diminishing bladder stress incontinence due to hormone loss. The use of the pet sex hormone loss treatment composition may also delay the onset of canine cognitive impairment.

In a preferred embodiment, the pet sex hormone loss treatment composition may be manufactured by compounding or otherwise mixing the ingredients and excipients. Such excipients are substances that are of little or no therapeutic value, but useful in the manufacture and compounding of the compositions. Exemplary excipients include, but limited to, celluloses (e.g. cellulose, microcrystalline cellulose, hydroxypropyl cellulose, hydroxypropyl methylcellulose, etc.), starches—in any form (plain, modified, pregelatinized, etc.) from (e.g. corn/maize, rice, pea, tapioca, wheat, etc.), mineral salts (e.g. carbonates, phosphates, etc.), silicates (calcium or magnesium salts), sugar alcohols/compounds (e.g. lactose, dextrin, dextrose, glucose, sucrose, sorbitol, etc.), gelatin (bovine, porcine, piscine), disintegrants (e.g. croscarmellose sodium, crosspovidone, sodium starch glycolate, etc.), stearic acid and its salts (e.g. calcium or magnesium stearate), natural or artificial colorants (e.g. caramel, FD&C colors, etc.) and combinations thereof. The pet sex hormone loss treatment composition may be either water or lipid soluble. Preferably, the pet sex hormone loss treatment composition may be water soluble. The pet sex hormone loss treatment composition may also be formulated into the form of dietary and nutritional supplement. Furthermore, the pet sex hormone loss treatment composition may also be manufactured into a kit, comprising some ingredients of the pet sex hormone loss treatment composition formulated into one dosage form and other ingredients into one same dosage form, or one or more different dosage forms.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further any specific numbers noted herein are only examples: alternative implementations may employ differing values or ranges.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A pet sex hormone loss treatment composition for treating pets that have been spayed or neutered comprising:
one or more amino acids; a genistein alternative selected from the group consisting of a SERM, genistein, aglycon genistine, hormone receptive modulators, phosphordiesterase (PDE) inhibitors of types 1, 2, 3 and 4, anti-neoplastic tyrosine kinase inhibitors, and combinations thereof; an icariin alternative selected from the group consisting of icarosides 1 or 2, PDE-4 inhibitors, PDE-5 inhibitors, derivatives of the epimedium species including icarrocides I and II, and combinations thereof, and a SARM, all in amounts to effectively treat sex hormone loss in the treated pet.

2. The pet sex hormone loss treatment composition for treating pets that have been spayed or neutered of claim 1 wherein the amino acid is N-acetyl cysteine.

3. The pet sex hormone loss treatment composition for treating pets that have been spayed or neutered of claim 1 wherein the genistein alternative is genistein or aglycon genistine.

4. The pet sex hormone loss treatment composition for treating pets that have been spayed or neutered of claim 1 wherein the icariin alternative is icariin or icarosides 1 or 2.

5. The pet sex hormone loss treatment composition for treating pets that have been spayed or neutered of claim 1 wherein the SARM is Momordica charantia L.

6. The pet sex hormone loss treatment composition for treating pets that have been spayed or neutered of claim 1 further comprising an ingredient selected from the group consisting of resveratrol, vitamin E, vitamin C, selenium, vitamin A, alpha-lipoic acid, lutein, inactive substances and combinations thereof.

7. A method for a spayed or neutered pet to mitigate sex hormone loss, the method comprising:
providing a pet sex hormone loss treatment composition in pet food, pet treats or pet drinking water; and
causing the animal to ingest the pet sex hormone loss treatment composition such that the pet sex hormone loss treatment composition reduces the sex hormone loss caused by spaying and neutering,
wherein the pet sex hormone loss treatment composition comprises one or more amino acids; a genistein alternative selected from the group consisting of a SERM, genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof, and at least one SARM.

8. The method of claim 7, wherein the pet sex hormone loss treatment composition further comprises an ingredient selected from the group consisting of resveratrol, vitamin E, vitamin C, L-selenomethionine, vitamin A, alpha-lipoic acid, lutein, inactive substances and combinations thereof

9. A method of treating a spayed or neutered animal, the method comprising causing the animal to ingest or intake a composition comprising of an effective amount one or more amino acids; a SERM, genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; an icariin alternative selected from the group consisting of icarosides 1 or 2, PDE-4 inhibitors, PDE-5 inhibitors, derivatives of the epimedium species including icarrocides I and II, and combinations thereof; and a SARM, whereby sex hormone loss in the animal is mitigated.
